(19) United States
(12) Patent Application Publication
(10) Pub. No.: US 2025/0134892 A1
(43) Pub. Date: May 1, 2025

(54) COMBINATION OF COMPOUNDS FOR TREATING VASCULAR DISEASES COMPRISING PDE5 INHIBITOR, ARGININE AND N-ACETYLCYSTEINE

(71) Applicant: Future Pharmtech Inc., Taipei City (TW)

(72) Inventors: Ming Wei CHAO, Taipei City (TW); Chin Hung LIN, Taipei City (TW); Po Tung CHEN, Taipei City (TW)

(21) Appl. No.: 18/836,532

(22) PCT Filed: Feb. 9, 2023
(86) PCT No.: PCT/GB2023/050296

§ 371 Date: Aug. 7, 2024

(30) Foreign Application Priority Data
Feb. 9, 2022 (TW) 111104639

(51) Int. Cl. A61K 31/519; A61K 31/197; A61K 31/198; A61K 45/06

(52) U.S. Cl. CPC A61K 31/519; A61K 31/197; A61K 31/198; A61K 45/06

(57) ABSTRACT

The present disclosure relates to the technical field of medicaments. Particularly, the present disclosure provides a pharmaceutical combination/composition comprising a phosphodiesterase type 5 (PDE5) inhibitor, arginine, and N-acetylcysteine and its applications in treating cardiovascular diseases and erectile dysfunction.

### RELATED APPLICATION

This application is a 371 National Phase application based upon PCT/GB23/50296, filed Feb. 9, 2023, which claims the benefit of Taiwan (R.O.C.) patent application No. 111104639, filed on 9 Feb. 2022, the contents thereof are of which are incorporated by reference in their entirety.

### FIELD

The present disclosure relates to the technical field of medicaments. Particularly, the present disclosure provides a pharmaceutical combination/composition comprising a phosphodiesterase type 5 (PDE5) inhibitor, arginine, and N-acetylcysteine and its applications in treating cardiovascular diseases and erectile dysfunction.

### BACKGROUND OF THE INVENTION

Cardiovascular disease is a very serious health problem in modern society. With the development of industry, in addition to high-salt and high-fat diets, the possibility of cardiovascular damage increases year over year due to the intake of various environmental pollutants or chemical compounds in daily life.

Erectile dysfunction (ED) is a problem to which much attention has been paid in recent years. In addition to psychological factors, erectile dysfunction may be caused by abnormal bodily functions. There has been a significant amount of research on the use of drugs to treat and relieve ED.

A phosphodiesterase type 5 inhibitor (PDE5 inhibitor) is a vasodilating drug that works by blocking the degradative action of cGMP-specific phosphodiesterase type 5 (PDE5) on cyclic GMP in the smooth muscle cells lining the blood vessels supplying various tissues. Part of the physiological process of vasodilatation involves the release of nitric oxide (NO) by vascular endothelial cells which then diffuses to nearby vascular smooth muscle cells. There, NO activates soluble guanylate cyclase which converts guanosine triphosphate (GTP) to cyclic guanosine monophosphate (cGMP), the main effector of the system. PDE5 inhibitors prolong the action of cGMP by inhibiting its degradation by the enzyme PDE5, which is found throughout the body (Goldstein I, Lue T F, Padma-Nathan H, Rosen R C, Steers W D, Wicker P A (May 1998). “Oral sildenafil in the treatment of erectile dysfunction. Sildenafil Study Group”, The New England Journal of Medicine. 338 (20): 1397-404). PDE5 inhibitors are contraindicated with alpha-blockers, soluble guanylate cyclase stimulators, or nitrate medications, which can cause blood pressure to be too low. The PDE5 inhibitor may cause headaches, stomachaches, diarrhea, flushing, dizziness, weakness, pruritus, erythema and the like after administration.

To date, there is still a need for developing a safer and more efficacious pharmaceutical composition or combination useful for treating or preventing cardiovascular diseases and ED.

### SUMMARY OF THE INVENTION

The present disclosure relates to a PDE5 inhibitor or its pharmaceutically acceptable salt in combination with arginine and/or N-acetylcysteine (NAC), which surprisingly increases the reaction rate of the PDE5 inhibitor. In particular, the reaction time is shortened, and endogenous free radicals produced by rapid vasodilatation are further scavenged so that the blood vessels may be protected.

In one embodiment, the present disclosure relates to a pharmaceutical combination or composition comprising a phosphodiesterase type 5 (PDE5) inhibitor or a pharmaceutically acceptable salt thereof, arginine, and N-acetylcysteine.

In one embodiment, the present disclosure relates to a pharmaceutical or composition comprising a PDE5 inhibitor or a pharmaceutically acceptable salt thereof, arginine, and N-acetylcysteine and a pharmaceutically acceptable excipient.

In one embodiment, the PDE5 inhibitor in the present disclosure is selected from sildenafil, tadalafil, and vardenafil and or a pharmaceutically acceptable salt thereof.

In one embodiment, the amount of the PDE5 inhibitor in the pharmaceutical combination or composition as described herein ranges from about 0.1% (w/w) to about 50% (w/w). Certain embodiments of the amount of the PDE5 inhibitor include, but are not limited to, about 0.1% (w/w) to about 45% (w/w), about 0.1% (w/w) to about 40% (w/w), about 0.1% (w/w) to about 35% (w/w), about 0.1% (w/w) to about 30% (w/w), about 0.1% (w/w) to about 25% (w/w), about 0.1% (w/w) to about 20% (w/w), about 0.1% (w/w) to about 15% (w/w), about 0.1% (w/w) to about 10% (w/w), about 0.1% (w/w) to about 5% (w/w), about 0.1% (w/w) to about 2.5% (w/w), about 0.1% (w/w) to about 1% (w/w), about 0.5% (w/w) to about 50% (w/w), about 0.5% (w/w) to about 45% (w/w), about 0.5% (w/w) to about 40% (w/w), about 0.5% (w/w) to about 35% (w/w), about 0.5% (w/w) to about 30% (w/w), about 0.5% (w/w) to about 25% (w/w), about 0.5% (w/w) to about 20% (w/w), about 0.5% (w/w) to about 15% (w/w), about 0.5% (w/w) to about 10% (w/w), about 0.5% (w/w) to about 5% (w/w), about 0.5% (w/w) to about 2.5% (w/w), about 0.5% (w/w) to about 1% (w/w), about 1.0% (w/w) to about 50% (w/w), about 1.0% (w/w) to about 45% (w/w), about 1.0% (w/w) to about 40% (w/w), about 1.0% (w/w) to about 35% (w/w), about 1.0% (w/w) to about 30% (w/w), about 1.0% (w/w) to about 25% (w/w), about 1.0% (w/w) to about 20% (w/w), about 1.0% (w/w) to about 15% (w/w), about 1.0% (w/w) to about 10% (w/w), about 1.0% (w/w) to about 5% (w/w), about 5% (w/w) to about 50% (w/w), about 5% (w/w) to about 45% (w/w), about 5% (w/w) to about 40% (w/w), about 5% (w/w) to about 35% (w/w), about 5% (w/w) to about 30% (w/w), about 5% (w/w) to about 25% (w/w), about 5% (w/w) to about 20% (w/w), about 5% (w/w) to about 15% (w/w), about 5% (w/w) to about 10% (w/w), about 10% (w/w) to about 50% (w/w), about 10% (w/w) to about 45% (w/w), about 10% (w/w) to about 40% (w/w), about 10% (w/w) to about 35% (w/w), about 10% (w/w) to about 30% (w/w), about 10% (w/w) to about 25% (w/w), about 10% (w/w) to about 20% (w/w), about 15% (w/w) to about 50% (w/w), about 15% (w/w) to about 45% (w/w), about 15% (w/w) to about 40% (w/w), about 15% (w/w) to about 35% (w/w), about 15% (w/w) to about 30% (w/w), about 15% (w/w) to about 25% (w/w), about 20% (w/w) to about 50% (w/w), about 20% (w/w) to about 45% (w/w), about 20% (w/w) to about 40% (w/w), about 20% (w/w) to about 35% (w/w), about 20% (w/w) to about 30% (w/w), about 20% (w/w) to about 25% (w/w), about 25% (w/w) to about 50% (w/w), about 25% (w/w) to about 45% (w/w), about 25% (w/w) to about 40% (w/w), about 25% (w/w) to about 35% (w/w), about 30% (w/w) to about 50% (w/w), about 30% (w/w) to about 45% (w/w), about 30% (w/w) to about 40% (w/w), about 35% (w/w) to about 50% (w/w), about 35% (w/w) to about 45% (w/w), about 40% (w/w) to about 50% (w/w) and about 40% (w/w) to about 45% (w/w).

In one embodiment, the amount of arginine in the pharmaceutical combination or composition as described herein ranges from about 4.0% (w/w) to about 80% (w/w). Certain embodiments of the amount of arginine include, but are not limited to, about 4.0% (w/w) to about 75% (w/w), about 4.0% (w/w) to about 70% (w/w), about 4.0% (w/w) to about 65% (w/w), about 4.0% (w/w) to about 60% (w/w), about 4.0% (w/w) to about 55% (w/w), about 4.0% (w/w) to about 50% (w/w), about 4.0% (w/w) to about 45% (w/w), about 4.0% (w/w) to about 40% (w/w), about 4.0% (w/w) to about 35% (w/w), about 4.0% (w/w) to about 30% (w/w), about 4.0% (w/w) to about 25% (w/w), about 4.0% (w/w) to about 20% (w/w), about 4.0% (w/w) to about 15% (w/w), about 4.0% (w/w) to about 10% (w/w), about 10.0% (w/w) to about 80% (w/w), about 10.0% (w/w) to about 75% (w/w), about 10.0% (w/w) to about 70% (w/w), about 10.0% (w/w) to about 65% (w/w), about 10.0% (w/w) to about 60% (w/w), about 10.0% (w/w) to about 55% (w/w), about 10.0% (w/w) to about 50% (w/w), about 10.0% (w/w) to about 45% (w/w), about 10.0% (w/w) to about 40% (w/w), about 10.0% (w/w) to about 35% (w/w), about 10.0% (w/w) to about 30% (w/w), about 10.0% (w/w) to about 25% (w/w), about 10.0% (w/w) to about 20% (w/w), about 10.0% (w/w) to about 15% (w/w), about 15.0% (w/w) to about 80% (w/w), about 15.0% (w/w) to about 75% (w/w), about 15.0% (w/w) to about 70% (w/w), about 15.0% (w/w) to about 65% (w/w), about 15.0% (w/w) to about 60% (w/w), about 15.0% (w/w) to about 55% (w/w), about 15.0% (w/w) to about 50% (w/w), about 15.0% (w/w) to about 45% (w/w), about 15.0% (w/w) to about 40% (w/w), about 15.0% (w/w) to about 35% (w/w), about 15.0% (w/w) to about 30% (w/w), about 15.0% (w/w) to about 25% (w/w), about 15.0% (w/w) to about 20% (w/w), about 20.0% (w/w) to about 80% (w/w), about 20.0% (w/w) to about 75% (w/w), about 20.0% (w/w) to about 70% (w/w), about 20.0% (w/w) to about 65% (w/w), about 20.0% (w/w) to about 60% (w/w), about 20.0% (w/w) to about 55% (w/w), about 20.0% (w/w) to about 50% (w/w), about 20.0% (w/w) to about 45% (w/w), about 20.0% (w/w) to about 40% (w/w), about 20.0% (w/w) to about 35% (w/w), about 20.0% (w/w) to about 30% (w/w), about 20.0% (w/w) to about 25% (w/w), about 25.0% (w/w) to about 80% (w/w), about 25.0% (w/w) to about 75% (w/w), about 25.0% (w/w) to about 70% (w/w), about 25.0% (w/w) to about 65% (w/w), about 25.0% (w/w) to about 60% (w/w), about 25.0% (w/w) to about 55% (w/w), about 25.0% (w/w) to about 50% (w/w), about 25.0% (w/w) to about 45% (w/w), about 25.0% (w/w) to about 40% (w/w), about 25.0% (w/w) to about 35% (w/w), about 25.0% (w/w) to about 30% (w/w), about 30.0% (w/w) to about 80% (w/w), about 30.0% (w/w) to about 75% (w/w), about 30.0% (w/w) to about 70% (w/w), about 30.0% (w/w) to about 65% (w/w), about 30.0% (w/w) to about 60% (w/w), about 30.0% (w/w) to about 55% (w/w), about 30.0% (w/w) to about 50% (w/w), about 30.0% (w/w) to about 45% (w/w), about 30.0% (w/w) to about 40% (w/w), about 30.0% (w/w) to about 35% (w/w), about 35.0% (w/w) to about 80% (w/w), about 35.0% (w/w) to about 75% (w/w), about 35.0% (w/w) to about 70% (w/w), about 35.0% (w/w) to about 65% (w/w), about 35.0% (w/w) to about 60% (w/w), about 35.0% (w/w) to about 55% (w/w), about 35.0% (w/w) to about 50% (w/w), about 35.0% (w/w) to about 45% (w/w), about 35.0% (w/w) to about 40% (w/w), about 40.0% (w/w) to about 80% (w/w), about 40.0% (w/w) to about 75% (w/w), about 40.0% (w/w) to about 70% (w/w), about 40.0% (w/w) to about 65% (w/w), about 40.0% (w/w) to about 60% (w/w), about 40.0% (w/w) to about 55% (w/w), about 40.0% (w/w) to about 50% (w/w), about 40.0% (w/w) to about 45% (w/w), about 45.0% (w/w) to about 80% (w/w), about 45.0% (w/w) to about 75% (w/w), about 45.0% (w/w) to about 70% (w/w), about 45.0% (w/w) to about 65% (w/w), about 45.0% (w/w) to about 60% (w/w), about 45.0% (w/w) to about 55% (w/w), about 45.0% (w/w) to about 50% (w/w), about 50.0% (w/w) to about 80% (w/w), about 50.0% (w/w) to about 75% (w/w), about 50.0% (w/w) to about 70% (w/w), about 50.0% (w/w) to about 65% (w/w), about 50.0% (w/w) to about 60% (w/w), about 50.0% (w/w) to about 55% (w/w), about 55.0% (w/w) to about 80% (w/w), about 55.0% (w/w) to about 75% (w/w), about 55.0% (w/w) to about 70% (w/w), about 55.0% (w/w) to about 65% (w/w), about 55.0% (w/w) to about 60% (w/w), about 60.0% (w/w) to about 80% (w/w), about 60.0% (w/w) to about 75% (w/w), about 60.0% (w/w) to about 70% (w/w), about 60.0% (w/w) to about 65% (w/w), about 65.0% (w/w) to about 80% (w/w), about 65.0% (w/w) to about 75% (w/w), about 65.0% (w/w) to about 70% (w/w), about 70.0% (w/w) to about 80% (w/w), about 70.0% (w/w) to about 75% (w/w), or about 75.0% (w/w) to about 80% (w/w).

In one embodiment, the amount of N-acetylcysteine in the pharmaceutical combination or composition as described herein ranges from about 4% (w/w) to about 80% (w/w). Certain embodiments of the amount of arginine include, but are not limited to, about 4.0% (w/w) to about 75% (w/w), about 4.0% (w/w) to about 70% (w/w), about 4.0% (w/w) to about 65% (w/w), about 4.0% (w/w) to about 60% (w/w), about 4.0% (w/w) to about 55% (w/w), about 4.0% (w/w) to about 50% (w/w), about 4.0% (w/w) to about 45% (w/w), about 4.0% (w/w) to about 40% (w/w), about 4.0% (w/w) to about 35% (w/w), about 4.0% (w/w) to about 30% (w/w), about 4.0% (w/w) to about 25% (w/w), about 4.0% (w/w) to about 20% (w/w), about 4.0% (w/w) to about 15% (w/w), about 4.0% (w/w) to about 10% (w/w), about 10.0% (w/w) to about 80% (w/w), about 10.0% (w/w) to about 75% (w/w), about 10.0% (w/w) to about 70% (w/w), about 10.0% (w/w) to about 65% (w/w), about 10.0% (w/w) to about 60% (w/w), about 10.0% (w/w) to about 55% (w/w), about 10.0% (w/w) to about 50% (w/w), about 10.0% (w/w) to about 45% (w/w), about 10.0% (w/w) to about 40% (w/w), about 10.0% (w/w) to about 35% (w/w), about 10.0% (w/w) to about 30% (w/w), about 10.0% (w/w) to about 25% (w/w), about 10.0% (w/w) to about 20% (w/w), about 10.0% (w/w) to about 15% (w/w), about 15.0% (w/w) to about 80% (w/w), about 15.0% (w/w) to about 75% (w/w), about 15.0% (w/w) to about 70% (w/w), about 15.0% (w/w) to about 65% (w/w), about 15.0% (w/w) to about 60% (w/w), about 15.0% (w/w) to about 55% (w/w), about 15.0% (w/w) to about 50% (w/w), about 15.0% (w/w) to about 45% (w/w), about 15.0% (w/w) to about 40% (w/w), about 15.0% (w/w) to about 35% (w/w), about 15.0% (w/w) to about 30% (w/w), about 15.0% (w/w) to about 25% (w/w), about 15.0% (w/w) to about 20% (w/w), about 20.0% (w/w) to about 80% (w/w), about 20.0% (w/w) to about 75% (w/w), about 20.0% (w/w) to about 70% (w/w), about 20.0% (w/w) to about 65% (w/w), about 20.0% (w/w) to about 60% (w/w), about 20.0% (w/w) to about 55% (w/w), about 20.0% (w/w) to about 50% (w/w), about 20.0% (w/w) to about 45% (w/w), about 20.0% (w/w) to about 40% (w/w), about 20.0% (w/w) to about 35% (w/w), about 20.0% (w/w) to about 30% (w/w), about 20.0% (w/w) to about 25% (w/w), about 25.0% (w/w) to about 80% (w/w), about 25.0% (w/w) to about 75% (w/w), about 25.0% (w/w) to about 70% (w/w), about 25.0% (w/w) to about 65% (w/w), about 25.0% (w/w) to about 60% (w/w), about 25.0% (w/w) to about 55% (w/w), about 25.0% (w/w) to about 50% (w/w), about 25.0% (w/w) to about 45% (w/w), about 25.0% (w/w) to about 40% (w/w), about 25.0% (w/w) to about 35% (w/w), about 25.0% (w/w) to about 30% (w/w), about 30.0% (w/w) to about 80% (w/w), about 30.0% (w/w) to about 75% (w/w), about 30.0% (w/w) to about 70% (w/w), about 30.0% (w/w) to about 65% (w/w), about 30.0% (w/w) to about 60% (w/w), about 30.0% (w/w) to about 55% (w/w), about 30.0% (w/w) to about 50% (w/w), about 30.0% (w/w) to about 45% (w/w), about 30.0% (w/w) to about 40% (w/w), about 30.0% (w/w) to about 35% (w/w), about 35.0% (w/w) to about 80% (w/w), about 35.0% (w/w) to about 75% (w/w), about 35.0% (w/w) to about 70% (w/w), about 35.0% (w/w) to about 65% (w/w), about 35.0% (w/w) to about 60% (w/w), about 35.0% (w/w) to about 55% (w/w), about 35.0% (w/w) to about 50% (w/w), about 35.0% (w/w) to about 45% (w/w), about 35.0% (w/w) to about 40% (w/w), about 40.0% (w/w) to about 80% (w/w), about 40.0% (w/w) to about 75% (w/w), about 40.0% (w/w) to about 70% (w/w), about 40.0% (w/w) to about 65% (w/w), about 40.0% (w/w) to about 60% (w/w), about 40.0% (w/w) to about 55% (w/w), about 40.0% (w/w) to about 50% (w/w), about 40.0% (w/w) to about 45% (w/w), about 45.0% (w/w) to about 80% (w/w), about 45.0% (w/w) to about 75% (w/w), about 45.0% (w/w) to about 70% (w/w), about 45.0% (w/w) to about 65% (w/w), about 45.0% (w/w) to about 60% (w/w), about 45.0% (w/w) to about 55% (w/w), about 45.0% (w/w) to about 50% (w/w), about 50.0% (w/w) to about 80% (w/w), about 50.0% (w/w) to about 75% (w/w), about 50.0% (w/w) to about 70% (w/w), about 50.0% (w/w) to about 65% (w/w), about 50.0% (w/w) to about 60% (w/w), about 50.0% (w/w) to about 55% (w/w), about 55.0% (w/w) to about 80% (w/w), about 55.0% (w/w) to about 75% (w/w), about 55.0% (w/w) to about 70% (w/w), about 55.0% (w/w) to about 65% (w/w), about 55.0% (w/w) to about 60% (w/w), about 60.0% (w/w) to about 80% (w/w), about 60.0% (w/w) to about 75% (w/w), about 60.0% (w/w) to about 70% (w/w), about 60.0% (w/w) to about 65% (w/w), about 65.0% (w/w) to about 80% (w/w), about 65.0% (w/w) to about 75% (w/w), about 65.0% (w/w) to about 70% (w/w), about 70.0% (w/w) to about 80% (w/w), about 70.0% (w/w) to about 75% (w/w), or about 75.0% (w/w) to about 80% (w/w).

In one embodiment, the amounts of the PDE5 inhibitor, arginine and N-acetylcysteine range from about 0.1% (w/w) to about 50% (w/w), about 4.0% (w/w) to about 80% (w/w) and about 4% (w/w) to about 80% (w/w), respectively. Certain embodiments of the amount of the PDE5 inhibitor, arginine, and N-acetylcysteine is in the range as described herein.

In one embodiment, the amount of the PDE5 inhibitor in the pharmaceutical combination or composition as described herein ranges from about 0.5 mg to about 250 mg. Certain embodiments of the amount of the PDE5 inhibitor include, but are not limited to, about 0.5 mg to about 250 mg, about 0.5 mg to about 240 mg, about 0.5 mg to about 230 mg, about 0.5 mg to about 220 mg, 0.5 mg to about 210 mg, about 0.5 mg to about 200 mg, about 0.5 mg to about 190 mg, about 0.5 mg to about 180 mg, about 0.5 mg to about 170 mg, about 0.5 mg to about 160 mg, about 0.5 mg to about 150 mg, about 0.5 mg to about 140 mg, about 0.5 mg to about 130 mg, about 0.5 mg to about 120 mg, about 0.5 mg to about 110 mg, about 0.5 mg to about 100 mg, about 0.5 mg to about 90 mg, about 0.5 mg to about 80 mg, about 0.5 mg to about 70 mg, about 0.5 mg to about 60 mg, about 0.5 mg to about 50 mg, about 0.5 mg to about 40 mg, about 0.5 mg to about 30 mg, about 0.5 mg to about 20 mg, about 0.5 mg to about 10 mg, about 0.5 mg to about 5 mg, about 0.5 mg to about 1 mg, about 1 mg to about 250 mg, about 1 mg to about 240 mg, about 1 mg to about 230 mg, about 1 mg to about 220 mg, about 1 mg to about 210 mg, about 1 mg to about 200 mg, about 1 mg to about 190 mg, about 1 mg to about 180 mg, about 1 mg to about 170 mg, about 1 mg to about 160 mg, about 1 mg to about 150 mg, about 1 mg to about 140 mg, about 1 mg to about 130 mg, about 1 mg to about 120 mg, about 1 mg to about 110 mg, about 1 mg to about 100 mg, about 1 mg to about 90 mg, about 1 mg to about 80 mg, about 1 mg to about 70 mg, about 1 mg to about 60 mg, about 1 mg to about 50 mg, about 1 mg to about 40 mg, about 1 mg to about 30 mg, about 1 mg to about 20 mg, about 1 mg to about 10 mg, about 1 mg to about 5 mg, about 5 mg to about 250 mg, about 5 mg to about 240 mg, about 5 mg to about 230 mg, about 5 mg to about 220 mg, about 5 mg to about 210 mg, about 5 mg to about 200 mg, about 5 mg to about 190 mg, about 5 mg to about 180 mg, about 5 mg to about 170 mg, about 5 mg to about 160 mg, about 5 mg to about 150 mg, about 5 mg to about 140 mg, about 5 mg to about 130 mg, about 5 mg to about 120 mg, about 5 mg to about 110 mg, about 5 mg to about 100 mg, about 5 mg to about 90 mg, about 5 mg to about 80 mg, about 5 mg to about 70 mg, about 5 mg to about 60 mg, about 5 mg to about 50 mg, about 5 mg to about 40 mg, about 5 mg to about 30 mg, about 5 mg to about 20 mg, about 5 mg to about 10 mg, about 10 mg to about 250 mg, about 10 mg to about 240 mg, about 10 mg to about 230 mg, about 10 mg to about 220 mg, about 10 mg to about 210 mg, about 10 mg to about 200 mg, about 10 mg to about 190 mg, about 10 mg to about 180 mg, about 10 mg to about 170 mg, about 10 mg to about 160 mg, about 10 mg to about 150 mg, about 10 mg to about 140 mg, about 10 mg to about 130 mg, about 10 mg to about 120 mg, about 10 mg to about 110 mg, about 10 mg to about 100 mg, about 10 mg to about 90 mg, about 10 mg to about 80 mg, about 10 mg to about 70 mg, about 10 mg to about 60 mg, about 10 mg to about 50 mg, about 10 mg to about 40 mg, about 10 mg to about 30 mg, about 10 mg to about 20 mg, about 20 mg to about 250 mg, about 20 mg to about 240 mg, about 20 mg to about 230 mg, about 20 mg to about 220 mg, about 20 mg to about 210 mg, about 20 mg to about 200 mg, about 20 mg to about 190 mg, about 20 mg to about 180 mg, about 20 mg to about 170 mg, about 20 mg to about 160 mg, about 20 mg to about 150 mg, about 20 mg to about 140 mg, about 20 mg to about 130 mg, about 20 mg to about 120 mg, about 20 mg to about 110 mg, about 20 mg to about 100 mg, about 20 mg to about 90 mg, about 20 mg to about 80 mg, about 20 mg to about 70 mg, about 20 mg to about 60 mg, about 20 mg to about 50 mg, about 20 mg to about 40 mg, about 20 mg to about 30 mg, about 30 mg to about 250 mg, 30 mg to about 240 mg, 30 mg to about 230 mg, 30 mg to about 220 mg, 30 mg to about 210 mg, about 30 mg to about 200 mg, about 30 mg to about 190 mg, about 30 mg to about 180 mg, about 30 mg to about 170 mg, about 30 mg to about 160 mg, about 30 mg to about 150 mg, about 30 mg to about 140 mg, about 30 mg to about 130 mg, about 30 mg to about 120 mg, about 30 mg to about 110 mg, about 30 mg to about 100 mg, about 30 mg to about 90 mg, about 30 mg to about 80 mg, about 30 mg to about 70 mg, about 30 mg to about 60 mg, about 30 mg to about 50 mg, about 30 mg to about 40 mg, about 40 mg to about 250 mg, about 40 mg to about 240 mg, about 40 mg to about 230 mg, about 40 mg to about 220 mg, about 40 mg to about 210 mg, about 40 mg to about 200 mg, about 40 mg to about 190 mg, about 40 mg to about 180 mg, about 40 mg to about 170 mg, about 40 mg to about 160 mg, about 40 mg to about 150 mg, about 40 mg to about 1 about 40 mg, about 40 mg to about 130 mg, about 40 mg to about 120 mg, about 40 mg to about 110 mg, about 40 mg to about 100 mg, about 40 mg to about 90 mg, about 40 mg to about 80 mg, about 40 mg to about 70 mg, about 40 mg to about 60 mg, about 40 mg to about 50 mg, about 50 mg to about 250 mg, about 50 mg to about 240 mg, about 50 mg to about 230 mg, about 50 mg to about 220 mg, about 50 mg to about 210 mg, about 50 mg to about 200 mg, about 50 mg to about 190 mg, about 50 mg to about 180 mg, about 50 mg to about 170 mg, about 50 mg to about 160 mg, about 50 mg to about 1 about 50 mg, about 50 mg to about 140 mg, about 50 mg to about 130 mg, about 50 mg to about 120 mg, about 50 mg to about 110 mg, about 50 mg to about 100 mg, about 50 mg to about 90 mg, about 50 mg to about 80 mg, about 50 mg to about 70 mg, about 50 mg to about 60 mg, about 60 mg to about 250 mg, about 60 mg to about 240 mg, about 60 mg to about 230 mg, about 60 mg to about 220 mg, about 60 mg to about 210 mg, about 60 mg to about 200 mg, about 60 mg to about 190 mg, about 60 mg to about 180 mg, about 60 mg to about 170 mg, about 60 mg to about 1 about 60 mg, about 60 mg to about 150 mg, about 60 mg to about 140 mg, about 60 mg to about 130 mg, about 60 mg to about 120 mg, about 60 mg to about 110 mg, about 60 mg to about 100 mg, about 60 mg to about 90 mg, about 60 mg to about 80 mg, about 60 mg to about 70 mg, about 70 mg to about 250 mg, about 70 mg to about 240 mg, about 70 mg to about 230 mg, about 70 mg to about 220 mg, about 70 mg to about 210 mg, about 70 mg to about 200 mg, about 70 mg to about 190 mg, about 70 mg to about 180 mg, about 70 mg to about 1 about 70 mg, about 70 mg to about 160 mg, about 70 mg to about 150 mg, about 70 mg to about 140 mg, about 70 mg to about 130 mg, about 70 mg to about 120 mg, about 70 mg to about 110 mg, about 70 mg to about 100 mg, about 70 mg to about 90 mg, about 70 mg to about 80 mg, about 80 mg to about 250 mg, about 80 mg to about 240 mg, about 80 mg to about 230 mg, about 80 mg to about 220 mg, about 80 mg to about 210 mg, about 80 mg to about 200 mg, about 80 mg to about 190 mg, about 80 mg to about 1 about 80 mg, about 80 mg to about 170 mg, about 80 mg to about 160 mg, about 80 mg to about 150 mg, about 80 mg to about 140 mg, about 80 mg to about 130 mg, about 80 mg to about 120 mg, about 80 mg to about 110 mg, about 80 mg to about 100 mg, about 80 mg to about 90 mg, about 90 mg to about 250 mg, 90 mg to about 240 mg, 90 mg to about 230 mg, 90 mg to about 220 mg, 90 mg to about 210 mg, 90 mg to about 200 mg, 90 mg to about 190 mg, 90 mg to about 180 mg, about 90 mg to about 170 mg, about 90 mg to about 160 mg, about 90 mg to about 150 mg, about 90 mg to about 140 mg, about 90 mg to about 130 mg, about 90 mg to about 120 mg, about 90 mg to about 110 mg, about 90 mg to about 100 mg, about 100 mg to about 250 mg, about 100 mg to about 240 mg, about 100 mg to about 230 mg, about 100 mg to about 220 mg, about 100 mg to about 210 mg, about 100 mg to about 200 mg, about 100 mg to about 190 mg, about 100 mg to about 180 mg, about 100 mg to about 170 mg, about 100 mg to about 160 mg, about 100 mg to about 150 mg, about 100 mg to about 140 mg, about 100 mg to about 130 mg, about 100 mg to about 120 mg, about 100 mg to about 110 mg, about 110 mg to about 250 mg, about 110 mg to about 240 mg, about 110 mg to about 230 mg, about 110 mg to about 220 mg, about 110 mg to about 210 mg, about 110 mg to about 200 mg, about 110 mg to about 190 mg, about 110 mg to about 180 mg, about 110 mg to about 170 mg, about 110 mg to about 160 mg, about 110 mg to about 150 mg, about 110 mg to about 140 mg, about 110 mg to about 130 mg, about 110 mg to about 120 mg, about 120 mg to about 250 mg, about 120 mg to about 240 mg, about 120 mg to about 230 mg, about 120 mg to about 220 mg, about 120 mg to about 210 mg, about 120 mg to about 200 mg, about 120 mg to about 190 mg, about 120 mg to about 180 mg, about 120 mg to about 170 mg, about 120 mg to about 160 mg, about 120 mg to about 150 mg, about 120 mg to about 140 mg, about 120 mg to about 130 mg, about 130 mg to about 250 mg, 130 mg to about 240 mg, about 130 mg to about 230 mg, about 130 mg to about 220 mg, about 130 mg to about 210 mg, about 130 mg to about 200 mg, about 130 mg to about 190 mg, about 130 mg to about 180 mg, about 130 mg to about 170 mg, about 130 mg to about 160 mg, about 130 mg to about 150 mg, about 130 mg to about 140 mg, about 130 mg to about 250 mg, about 130 mg to about 240 mg, about 130 mg to about 230 mg, about 130 mg to about 220 mg, about 130 mg to about 210 mg, about 130 mg to about 200 mg, about 130 mg to about 190 mg, about 130 mg to about 180 mg, about 130 mg to about 170 mg, about 130 mg to about 160 mg, about 130 mg to about 150 mg, about 130 mg to about 140 mg, about 140 mg to about 250 mg, about 140 mg to about 240 mg, about 140 mg to about 230 mg, about 140 mg to about 220 mg, about 140 mg to about 210 mg, about 140 mg to about 200 mg, about 140 mg to about 190 mg, about 140 mg to about 180 mg, about 140 mg to about 170 mg, about 140 mg to about 160 mg, about 140 mg to about 150 mg, about 150 mg to about 250 mg, about 150 mg to about 240 mg, about 150 mg to about 230 mg, about 150 mg to about 220 mg, about 150 mg to about 210 mg, about 150 mg to about 200 mg, about 150 mg to about 190 mg, about 150 mg to about 180 mg, about 150 mg to about 170 mg, about 150 mg to about 160 mg, about 200 mg to about 250 mg, about 200 mg to about 240 mg, about 200 mg to about 230 mg, about 200 mg to about 220 mg, about 200 mg to about 210 mg.

In one embodiment, the amount of arginine or N-acetylcysteine in the pharmaceutical combination or composition as described herein ranges from about 25 mg to about 400 mg. Certain embodiments of the amount of the arginine or N-acetylcysteine include, but are not limited to, about 25 mg to about 400 mg, about 25 mg to about 390 mg, about 25 mg to about 380 mg, about 25 mg to about 370 mg, about 25 mg to about 360 mg, about 25 mg to about 350 mg, about 25 mg to about 340 mg, about 25 mg to about 330 mg, about 25 mg to about 320 mg, about 25 mg to about 310 mg, about 25 mg to about 300 mg, about 25 mg to about 290 mg, about 25 mg to about 280 mg, about 25 mg to about 270 mg, about 25 mg to about 260 mg, about 25 mg to about 250 mg, about 25 mg to about 240 mg, about 25 mg to about 230 mg, about 25 mg to about 220 mg, about 25 mg to about 210 mg, about 25 mg to about 200 mg, about 25 mg to about 190 mg, about 25 mg to about 180 mg, about 25 mg to about 170 mg, about 25 mg to about 160 mg, about 25 mg to about 150 mg, about 25 mg to about 140 mg, about 25 mg to about 130 mg, about 25 mg to about 120 mg, about 25 mg to about 110 mg, about 25 mg to about 100 mg, about 25 mg to about 90 mg, about 25 mg to about 80 mg, about 25 mg to about 70 mg, about 25 mg to about 60 mg, about 25 mg to about 50 mg, about 25 mg to about 40 mg, about 25 mg to about 30 mg, about 30 mg to about 400 mg, about 30 mg to about 390 mg, about 30 mg to about 380 mg, about 30 mg to about 370 mg, about 30 mg to about 360 mg, about 30 mg to about 350 mg, about 30 mg to about 340 mg, about 30 mg to about 330 mg, about 30 mg to about 320 mg, about 30 mg to about 310 mg, about 30 mg to about 300 mg, about 30 mg to about 290 mg, about 30 mg to about 280 mg, about 30 mg to about 270 mg, about 30 mg to about 260 mg, about 30 mg to about 300 mg, about 30 mg to about 240 mg, about 30 mg to about 230 mg, about 30 mg to about 220 mg, about 30 mg to about 210 mg, about 30 mg to about 200 mg, about 30 mg to about 190 mg, about 30 mg to about 180 mg, about 30 mg to about 170 mg, about 30 mg to about 160 mg, about 30 mg to about 150 mg, about 30 mg to about 140 mg, about 30 mg to about 130 mg, about 30 mg to about 120 mg, about 30 mg to about 110 mg, about 30 mg to about 100 mg, about 30 mg to about 90 mg, about 30 mg to about 80 mg, about 30 mg to about 70 mg, about 30 mg to about 60 mg, about 30 mg to about 50 mg, about 30 mg to about 40 mg, about 40 mg to about 400 mg, about 40 mg to about 390 mg, about 40 mg to about 380 mg, about 40 mg to about 370 mg, about 40 mg to about 360 mg, about 40 mg to about 350 mg, about 40 mg to about 340 mg, about 40 mg to about 330 mg, about 40 mg to about 320 mg, about 40 mg to about 310 mg, about 40 mg to about 300 mg, about 40 mg to about 290 mg, about 40 mg to about 280 mg, about 40 mg to about 270 mg, about 40 mg to about 260 mg, about 40 mg to about 400 mg, about 40 mg to about 240 mg, about 40 mg to about 230 mg, about 40 mg to about 220 mg, about 40 mg to about 210 mg, about 40 mg to about 200 mg, about 40 mg to about 190 mg, about 40 mg to about 180 mg, about 40 mg to about 170 mg, about 40 mg to about 160 mg, about 40 mg to about 150 mg, about 40 mg to about 140 mg, about 40 mg to about 130 mg, about 40 mg to about 120 mg, about 40 mg to about 110 mg, about 40 mg to about 100 mg, about 40 mg to about 90 mg, about 40 mg to about 80 mg, about 40 mg to about 70 mg, about 40 mg to about 60 mg, about 40 mg to about 50 mg, about 50 mg to about 400 mg, about 50 mg to about 390 mg, about 50 mg to about 380 mg, about 50 mg to about 370 mg, about 50 mg to about 360 mg, about 50 mg to about 350 mg, about 50 mg to about 340 mg, about 50 mg to about 330 mg, about 50 mg to about 320 mg, about 50 mg to about 310 mg, about 50 mg to about 300 mg, about 50 mg to about 290 mg, about 50 mg to about 280 mg, about 50 mg to about 270 mg, about 50 mg to about 260 mg, about 50 mg to about 500 mg, about 50 mg to about 240 mg, about 50 mg to about 230 mg, about 50 mg to about 220 mg, about 50 mg to about 210 mg, about 50 mg to about 200 mg, about 50 mg to about 190 mg, about 50 mg to about 180 mg, about 50 mg to about 170 mg, about 50 mg to about 160 mg, about 50 mg to about 150 mg, about 50 mg to about 140 mg, about 50 mg to about 130 mg, about 50 mg to about 120 mg, about 50 mg to about 110 mg, about 50 mg to about 100 mg, about 50 mg to about 90 mg, about 50 mg to about 80 mg, about 50 mg to about 70 mg, about 50 mg to about 60 mg, about 75 mg to about 400 mg, about 75 mg to about 390 mg, about 75 mg to about 380 mg, about 75 mg to about 370 mg, about 75 mg to about 360 mg, about 75 mg to about 350 mg, about 75 mg to about 340 mg, about 75 mg to about 330 mg, about 75 mg to about 320 mg, about 75 mg to about 310 mg, about 75 mg to about 300 mg, about 75 mg to about 290 mg, about 75 mg to about 280 mg, about 75 mg to about 270 mg, about 75 mg to about 260 mg, about 75 mg to about 750 mg, about 75 mg to about 240 mg, about 75 mg to about 230 mg, about 75 mg to about 220 mg, about 75 mg to about 210 mg, about 75 mg to about 200 mg, about 75 mg to about 190 mg, about 75 mg to about 180 mg, about 75 mg to about 170 mg, about 75 mg to about 160 mg, about 75 mg to about 150 mg, about 75 mg to about 140 mg, about 75 mg to about 130 mg, about 75 mg to about 120 mg, about 75 mg to about 110 mg, about 75 mg to about 100 mg, about 75 mg to about 90 mg, about 75 mg to about 80 mg, about 100 mg to about 400 mg, about 100 mg to about 390 mg, about 100 mg to about 380 mg, about 100 mg to about 370 mg, about 100 mg to about 360 mg, about 100 mg to about 350 mg, about 100 mg to about 340 mg, about 100 mg to about 330 mg, about 100 mg to about 320 mg, about 100 mg to about 310 mg, about 100 mg to about 300 mg, about 100 mg to about 290 mg, about 100 mg to about 280 mg, about 100 mg to about 270 mg, about 100 mg to about 260 mg, about 100 mg to about 1000 mg, about 100 mg to about 240 mg, about 100 mg to about 230 mg, about 100 mg to about 220 mg, about 100 mg to about 210 mg, about 100 mg to about 200 mg, about 100 mg to about 190 mg, about 100 mg to about 180 mg, about 100 mg to about 170 mg, about 100 mg to about 160 mg, about 100 mg to about 150 mg, about 100 mg to about 140 mg, about 100 mg to about 130 mg, about 100 mg to about 120 mg, about 100 mg to about 110 mg, about 125 mg to about 400 mg, about 125 mg to about 390 mg, about 125 mg to about 380 mg, about 125 mg to about 370 mg, about 125 mg to about 360 mg, about 125 mg to about 350 mg, about 125 mg to about 340 mg, about 125 mg to about 330 mg, about 125 mg to about 320 mg, about 125 mg to about 310 mg, about 125 mg to about 300 mg, about 125 mg to about 290 mg, about 125 mg to about 280 mg, about 125 mg to about 270 mg, about 125 mg to about 260 mg, about 125 mg to about 1250 mg, about 125 mg to about 240 mg, about 125 mg to about 230 mg, about 125 mg to about 220 mg, about 125 mg to about 210 mg, about 125 mg to about 200 mg, about 125 mg to about 190 mg, about 125 mg to about 180 mg, about 125 mg to about 170 mg, about 125 mg to about 160 mg, about 125 mg to about 150 mg, about 125 mg to about 140 mg, about 125 mg to about 130 mg, about 150 mg to about 400 mg, about 150 mg to about 390 mg, about 150 mg to about 380 mg, about 150 mg to about 370 mg, about 150 mg to about 360 mg, about 150 mg to about 350 mg, about 150 mg to about 340 mg, about 150 mg to about 330 mg, about 150 mg to about 320 mg, about 150 mg to about 310 mg, about 150 mg to about 300 mg, about 150 mg to about 290 mg, about 150 mg to about 280 mg, about 150 mg to about 270 mg, about 150 mg to about 260 mg, about 150 mg to about 250 mg, about 150 mg to about 240 mg, about 150 mg to about 230 mg, about 150 mg to about 220 mg, about 150 mg to about 210 mg, about 150 mg to about 200 mg, about 150 mg to about 190 mg, about 150 mg to about 180 mg, about 150 mg to about 170 mg, about 150 mg to about 160 mg, about 175 mg to about 400 mg, about 175 mg to about 390 mg, about 175 mg to about 380 mg, about 175 mg to about 370 mg, about 175 mg to about 360 mg, about 175 mg to about 350 mg, about 175 mg to about 340 mg, about 175 mg to about 330 mg, about 175 mg to about 320 mg, about 175 mg to about 310 mg, about 175 mg to about 300 mg, about 175 mg to about 290 mg, about 175 mg to about 280 mg, about 175 mg to about 270 mg, about 175 mg to about 260 mg, about 175 mg to about 250 mg, about 175 mg to about 240 mg, about 175 mg to about 230 mg, about 175 mg to about 220 mg, about 175 mg to about 210 mg, about 175 mg to about 200 mg, about 175 mg to about 190 mg, about 175 mg to about 180 mg, about 200 mg to about 400 mg, about 200 mg to about 390 mg, about 200 mg to about 380 mg, about 200 mg to about 370 mg, about 200 mg to about 360 mg, about 200 mg to about 350 mg, about 200 mg to about 340 mg, about 200 mg to about 330 mg, about 200 mg to about 320 mg, about 200 mg to about 310 mg, about 200 mg to about 300 mg, about 200 mg to about 290 mg, about 200 mg to about 280 mg, about 200 mg to about 270 mg, about 200 mg to about 260 mg, about 200 mg to about 2000 mg, about 200 mg to about 240 mg, about 200 mg to about 230 mg, about 200 mg to about 220 mg, about 200 mg to about 210 mg, about 225 mg to about 400 mg, about 225 mg to about 390 mg, about 225 mg to about 380 mg, about 225 mg to about 370 mg, about 225 mg to about 360 mg, about 225 mg to about 350 mg, about 225 mg to about 340 mg, about 225 mg to about 330 mg, about 225 mg to about 320 mg, about 225 mg to about 310 mg, about 225 mg to about 300 mg, about 225 mg to about 290 mg, about 225 mg to about 280 mg, about 225 mg to about 270 mg, about 225 mg to about 260 mg, about 225 mg to about 250 mg, about 225 mg to about 240 mg, about 225 mg to about 230 mg, about 225 mg to about 220 mg, about 225 mg to about 210 mg, about 225 mg to about 200 mg, about 250 mg to about 400 mg, about 250 mg to about 390 mg, about 250 mg to about 380 mg, about 250 mg to about 370 mg, about 250 mg to about 360 mg, about 250 mg to about 350 mg, about 250 mg to about 340 mg, about 250 mg to about 330 mg, about 250 mg to about 320 mg, about 250 mg to about 310 mg, about 250 mg to about 300 mg, about 250 mg to about 290 mg, about 250 mg to about 280 mg, about 250 mg to about 270 mg, about 250 mg to about 260 mg, about 275 mg to about 400 mg, about 275 mg to about 390 mg, about 275 mg to about 380 mg, about 275 mg to about 370 mg, about 275 mg to about 360 mg, about 275 mg to about 350 mg, about 275 mg to about 340 mg, about 275 mg to about 330 mg, about 275 mg to about 320 mg, about 275 mg to about 310 mg, about 275 mg to about 300 mg, about 275 mg to about 290 mg, about 275 mg to about 280 mg, about 300 mg to about 400 mg, about 300 mg to about 390 mg, about 300 mg to about 380 mg, about 300 mg to about 370 mg, about 300 mg to about 360 mg, about 300 mg to about 350 mg, about 300 mg to about 340 mg, about 300 mg to about 330 mg, about 300 mg to about 320 mg, about 300 mg to about 310 mg, about 325 mg to about 400 mg, about 325 mg to about 390 mg, about 325 mg to about 380 mg, about 325 mg to about 370 mg, about 325 mg to about 360 mg, about 350 mg to about 350 mg, about 325 mg to about 340 mg, about 325 mg to about 330 mg, about 350 mg to about 400 mg, about 350 mg to about 390 mg, about 350 mg to about 380 mg, about 350 mg to about 370 mg, about 350 mg to about 360 mg, about 375 mg to about 400 mg, about 375 mg to about 390 mg, about 375 mg to about 380 mg.

In one embodiment, the pharmaceutical combination or composition as described herein comprises about 0.5 mg to about 250 mg of the PDE5 inhibitor, about 25 mg to about 400 mg of arginine and about 25 mg to about 400 mg of N-acetylcysteine. Certain embodiments of the pharmaceutical combination or composition as described herein have the amount of the PDE5 inhibitor, N-acetylcysteine, and arginine as disclosed herein.

In some embodiments, the amount of the PDE5 inhibitor in the pharmaceutical combination or composition as described herein is about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 mg, the amount of arginine is about 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, or 275 mg, and/or the amount of N-acetylcysteine is about 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, or 275 mg. In a certain embodiment, the amount of the PDE5 inhibitor is about 20 mg, the amount of arginine is about 250 mg, and/or the amount of the N-acetylcysteine is about 250 mg. In a certain embodiment, the amount of the PDE5 inhibitor is about 20 mg, the amount of arginine is about 250 mg, and the amount of the N-acetylcysteine is about 250 mg.

In one embodiment, the daily dose of the pharmaceutical combination or composition is twice as the dose of the PDE5 inhibitor, arginine, and/or N-acetyl cysteine as described herein. In one embodiment, the pharmaceutical combination or composition disclosed herein is administered twice a day, so that the daily dose of the pharmaceutical combination or composition is twice as the dose of the PDE5 inhibitor, arginine, and/or N-acetyl cysteine as described herein.

In some embodiments, daily dose of the PDE5 inhibitor in the pharmaceutical combination or composition disclosed herein is about 4 mg to about 100 mg. In some embodiments, daily dose of arginine in the pharmaceutical combination or composition disclosed herein is about 20 mg to about 1000 mg. In some embodiments, daily dose of N-acetylcysteine in the pharmaceutical combination or composition disclosed herein is about 20 mg to about 1000 mg. Certain embodiments of the pharmaceutical combination or composition disclosed herein have a daily dose of the PDE5 inhibitor being about 40 mg. Certain embodiments of the pharmaceutical combination or composition disclosed herein have a daily dose of arginine being about 500 mg. Certain embodiments of the pharmaceutical combination or composition disclosed herein have a daily dose of the N-acetylcysteine being about 500 mg. Certain embodiments of the pharmaceutical combination or composition disclosed herein have a daily dose of the PDE5 inhibitor being about 40 mg, a daily dose of the arginine being about 500 mg, and/or the daily dose of the N-acetylcysteine being about 500 mg.

In one embodiment, the pharmaceutical combination or composition as described herein is in a liquid formulation, and the concentration of sildenafil disclosed herein is from about 1 to 1000 nM, the concentration of arginine is 1 to 1000 μM, and/or the concentration of N-acetylcysteine is 0.1 to 100 mM in the compound combination.

In one embodiment, the combination or composition disclosed herein is fast-acting, and/or can be taken long term with fewer side effects.

In one embodiment, the combination or composition disclosed herein is in the form of a solid or liquid formulation; in particular, a capsule or a tablet or a solution for injection or oral administration.

The present disclosure also provides a method for treating a vascular disease in a subject in need thereof comprising administering a pharmaceutical composition or combination disclosed herein to the subject. Alternatively, the present disclosure relates to a pharmaceutical composition or combination disclosed herein for use in treating a vascular disease. Alternatively, the present invention discloses the use of a combination in preparation of a drug for treating vascular diseases; wherein the combination includes sildenafil, arginine and N-acetylcysteine.

In one embodiment, the NAC and arginine can enhance the effect of a PDE5 inhibitor and simultaneously reduce adverse effects of the PDE5 inhibitor. Accordingly, the present disclosure also provides a method for reducing adverse effects of a PDE5 inhibitor, comprising administering a combination or composition of NAC and arginine with the PDE5 inhibitor.

In one embodiment, the dosing amount or administration amount disclosed herein is an effective amount or a therapeutically effective amount.

In one embodiment, the vascular disease in the present disclosure is selected from erectile dysfunction, mountain sickness, pulmonary hypertension, vascular fibrosis, and vascular sclerosis.

In one embodiment, the PDE5 inhibitor or a pharmaceutically acceptable salt thereof, arginine, and N-acetylcysteine disclosed herein is administered concomitantly, sequentially, or separately.

In one embodiment, the PDE5 inhibitor or a pharmaceutically acceptable salt thereof disclosed herein is administered concomitantly with N-acetylcysteine.

In a further embodiment, the PDE5 inhibitor is sildenafil or sildenafil citrate.

In one embodiment, the combination or composition disclosed herein result in synergistic effect compared with a PDE5 inhibitor or a pharmaceutically acceptable salt thereof, e.g., sildenafil or sildenafil citrate, alone.

In one embodiment, the combination or composition disclosed herein is for oral administration.

In one embodiment, the combination or composition disclosed herein is administered once or twice or three times a day; or once, twice, three times, four times, five times, six times, or seven times a week. In certain embodiment, the pharmaceutical combination or composition disclosed herein is administered twice a day.

In one embodiment, the agents (PDE5 inhibitor, arginine, and NAC) in the combination or composition disclosed herein are derivatives of said agents, including, but not limited to a salt, pharmaceutically acceptable salt, solvate, hydrate, ester, tautomer, stereoisomer, enantiomer, or diastereomer thereof. Unless otherwise specified, the agents in the combination or composition disclosed herein cover the aforementioned derivatives.

### BRIEF DESCRIPTION OF THE DRAWINGS

FIG. 1 is a scale chart of a relationship between the NO release amount and treatment time in treatment of vascular endothelial cells with Sildenafil (SILD).

FIG. 2 is a scale chart of NO release amount of vascular endothelial cells after the vascular endothelial cells are treated with Sildenafil (SILD), Sildenafil+N-acetylcysteine (SN), and Sildenafil+arginine+N-acetylcysteine (SAN) for 10 min.

FIG. 3 is a scale chart of the expression amount of vascular endothelial nitric oxide synthase after the vascular endothelial cells are treated with Sildenafil (SILD), Sildenafil+N-acetylcysteine (SN), and Sildenafil+arginine+N-acetylcysteine (SAN) for 10 min.

FIG. 4 is a scale chart showing the relationship between the cGMP expression amount of smooth muscle cells and treatment time in the treatment of smooth muscle cells with Sildenafil (SILD).

FIG. 5 is a scale chart of the cGMP expression amount of smooth muscle cells after the smooth muscle cells are treated with Sildenafil (SILD), Sildenafil+N-acetylcysteine (SN), and Sildenafil+arginine+N-acetylcysteine (SAN) for 10 min.

FIG. 6 is a scale chart of vascular endothelial cell free radical content after vascular endothelial cells are treated with Sildenafil (SILD), Sildenafil+N-acetylcysteine (SN), and Sildenafil+arginine+N-acetylcysteine (SAN) for 10 min.

FIG. 7 is a scale chart of vascular layer fibrosis indexes after vascular endothelial cells are treated with Sildenafil (SILD), Sildenafil+N-acetylcysteine (SN), and Sildenafil+arginine+N-acetylcysteine (SAN) for 10 min.

FIG. 8 is a scale chart of vascular permeability after vascular endothelial cells are treated with Sildenafil (SILD), Sildenafil+N-acetylcysteine (SN), and Sildenafil+arginine+N-acetylcysteine (SAN) for 10 min.

### DETAILED DESCRIPTION OF THE INVENTION

As used herein, “treatment” refers to clinical intervention in an attempt to alter the natural course of the individual or cell being treated, and can be performed either for prophylaxis or during the course of clinical pathology. Desirable effects of treatment include preventing occurrence or recurrence of disease, alleviation of symptoms, diminishment of any direct or indirect pathological consequences of the disease, preventing or decreasing inflammation and/or tissue/organ damage, decreasing the rate of disease occurrence, amelioration or palliation of the disease state, and remission or improved prognosis.

The term “pharmaceutical” or “pharmaceutically acceptable” when used herein as an adjective, means substantially non-toxic and substantially non-deleterious to the recipient. By “pharmaceutical composition” it is further meant that the carrier, solvent, excipients and salt must be compatible with the active ingredient of the composition (e.g., a compound of the invention). It is understood by those of ordinary skill in this art that the terms “pharmaceutical formulation” and “pharmaceutical composition” are generally interchangeable, and they are so used for the purposes of this application.

An “individual” or a “subject” disclosed herein is a vertebrate. In certain embodiments, the vertebrate is a mammal. Mammals include, but are not limited to, farm animals (such as cows), sport animals, pets (such as cats, dogs, and horses), primates, mice and rats. In certain embodiments, the vertebrate is a human.

An “effective amount” disclosed herein refers to an amount which is effective, at certain dosages and for necessary periods of time, to achieve the desired therapeutic or prophylactic result. In one embodiment, the PDE5 inhibitor, arginine, and/or NAC in the combination or composition disclosed herein is in an effective amount.

A “therapeutically effective amount” of a substance/molecule of the present disclosure may vary according to factors such as the disease state, age, sex, and weight of the individual, and the ability of the substance/molecule, to elicit a desired response in the individual. A therapeutically effective amount is also one in which any toxic or detrimental effects of the substance/molecule are outweighed by the therapeutically beneficial effects. A “prophylactically effective amount” refers to an amount which is effective, at certain dosages and for necessary periods of time, to achieve the desired prophylactic result. Typically, but not necessarily, since a prophylactic dose is used in subjects prior to or at an earlier stage of disease, the prophylactically effective amount would be less than the therapeutically effective amount. In one embodiment, the PDE5 inhibitor, arginine, and/or NAC in the combination or composition disclosed herein is in a therapeutically effective amount.

Administration “in combination with” as disclosed herein includes simultaneous (concurrent) and consecutive administration in any order. As used herein, “combination therapy” or “in combination” includes the administration of a PDE5 inhibitor, arginine, and NAC as part of a specific treatment regimen intended to provide the beneficial effect from the co-action of these agents.

“Excipients” as used herein include pharmaceutically acceptable carriers or stabilizers that are nontoxic to the cell or mammal being exposed thereto at the dosages and concentrations employed. In one embodiment, a physiologically acceptable carrier is an aqueous pH buffered solution.

The term “About” as used herein indicates a variation of about ±1, 2, 3, 4, 5, 6, 7, 8, 9, or 10%.

The present disclosure surprisingly found that the combination or composition of a PDE5 inhibitor (preferably sildenafil), arginine and N-acetylcysteine can increase the reaction rate of the PDE5 inhibitor or decrease the reaction time of the PDE5 inhibitor, compared with administration of the PDE5 inhibitor alone, and/or significantly increases the expression of nitric oxide synthase in a cell. Accordingly, the present disclosure provides a pharmaceutical combination comprising a PDE5 inhibitor (preferably sildenafil), arginine and N-acetylcysteine and a method of using the combination or composition to effectively treat a vascular disease with fewer side effects.

Several phosphodiesterase type 5 (PDE5) inhibitors or their pharmaceutically acceptable salts, such as sildenafil (SLID), tadalafil, and vardenafil, have been approved by the health authorities to treat erectile dysfunction. It has been found in previous experiments that the mechanism causing penile erection is nitric oxide (NO) diffusing into smooth muscle cells of the penis from vascular endothelial cells to activate guanylate cyclase (GC), thereby increasing the generation of cyclic guanosine monophosphate (cGMP) and inhibiting the inflow of calcium ions which will reduce the concentration of calcium ions in the cells and relax the smooth muscle of the cavernous body; the cavernous body is then congested and erected due to a large amount of blood. For example, sildenafil plays a role in inhibiting the hydrolysis of the cGMP, so that the cGMP can continuously act to achieve the effect of congestion and erection. According to the package insert of sildenafil, sildenafil has no direct relaxant effect on isolated human corpus cavernosum, but enhances the effect of nitric oxide (NO) by inhibiting phosphodiesterase type 5 (PDE5), which is responsible for degradation of cGMP in the corpus cavernosum. However, sildenafil also has multiple side effects. It may cause headaches, stomachaches, diarrhea, flushing, dizziness, weakness, pruritus, erythema and the like after administration. The side effects mostly occur within several minutes after sildenafil is taken. If Sildenafil is taken long term, serious hypertension may occur, and problems of transient loss of vision, coronary sclerosis and the like may be increased. These side effects are generally thought to be caused by vasodilatation; and rapid vasodilatation may cause vascular endothelial cells to bear pressure from high blood flow, which will cause vascular endothelial cells to release endogenous free radicals, thereby causing the vascular cells to be damaged by the free radicals.

Arginine is a medicine which can be used as an oral tablet and an oral capsule. In the erection process, L-arginine can be catalyzed to generate nitric oxide by endothelial nitric oxide synthase, and the nitric oxide may be quickly released to a space between cavernous tissues, so that the amount of divalent calcium ions in the cavernous artery smooth muscle cells is reduced to cause smooth muscle relaxation and increase the blood flow.

N-acetylcysteine (NAC) has an antioxidant effect and is capable of promoting the conversion of glutathione disulfide (GSSG) into glutathione (GSH) in the cells, and the GSH is an indispensable element for synthesizing the most effective antioxidant enzyme in the body. In addition, it was reported that NAC has an inhibitory effect on inducible nitric oxide synthase (NOS) protein and NO production (Araki et al., N-acetylcysteine inhibits induction of nitric oxide synthase in 3T3-L1 adipocytes, J UOEH 29 (4): 417-429 (2007); Bergamini et al., N—acetylcysteine inhibits in vivo nitric oxide production by inducible nitric oxide synthase, NITRIC OXIDE: Biology and Chemistry Vol. 5, No. 4, pp. 349-360 (2001); Rota et al., N—acetylcysteine negatively modulates nitric oxide production in endotoxin-treated rats through inhibition of NF-κB activation; Antioxidants & Redox Signaling, Vol. 4, No. 1 (2004)). Therefore, NAC causes adverse effects in NO production, which is contrary to the effect brought by arginine.

However, the present disclosure unexpectedly found that the combination of NAC and arginine can enhance the effect of a PDE5 inhibitor and simultaneously reduce the adverse effects thereof.

In the pharmaceutical combination, examples of the amount or dose of the PDE5 inhibitor used herein include, but are not limited to, from about 1 mg to about 500 mg, from about 5 mg to about 490 mg, from about 5 mg to about 450 mg, from about 5 mg to about 400 mg, from about 5 mg to about 350 mg, from about 5 mg to about 300 mg, from about 5 mg to about 250 mg, from about 5 mg to about 200 mg, from about 5 mg to about 175 mg, from about 5 mg to about 150 mg, from about 5 mg to about 125 mg, from about 5 mg to about 100 mg, from about 5 mg to about 90 mg, from about 5 mg to about 80 mg, from about 5 mg to about 70 mg, from about 5 mg to about 60 mg, from about 5 mg to about 50 mg, from about 5 mg to about 40 mg, from about 5 mg to about 30 mg, from about 5 mg to about 20 mg, from about 5 mg to about 10 mg, from about 10 mg to about 480 mg, about 10 mg to about 470 mg, about 10 mg to about 460 mg, about 10 mg to about 450 mg, about 10 mg to about 440 mg, about 10 mg to about 430 mg, about 10 mg to about 420 mg, about 10 mg to about 410 mg, about 10 mg to about 400 mg, about 10 mg to about 390 mg, about 10 mg to about 380 mg, about 10 mg to about 370 mg, about 10 mg to about 360 mg, about 10 mg to about 350 mg, about 10 mg to about 340 mg, about 10 mg to about 330 mg, about 10 mg to about 320 mg, about 10 mg to about 310 mg, about 10 mg to about 300 mg, about 10 mg to about 290 mg, about 10 mg to about 280 mg, about 10 mg to about 270 mg, about 10 mg to about 260 mg, about 10 mg to about 250 mg, about 10 mg to about 240 mg, about 10 mg to about 230 mg, about 10 mg to about 220 mg, about 10 mg to about 210 mg, about 10 mg to about 200 mg, about 10 mg to about 190 mg, about 10 mg to about 180 mg, about 10 mg to about 170 mg, about 10 mg to about 160 mg, about 10 mg to about 150 mg, about 10 mg to about 140 mg, about 10 mg to about 130 mg, about 10 mg to about 120 mg, about 10 mg to about 110 mg, about 10 mg to about 100 mg, about 10 mg to about 90 mg, about 10 mg to about 80 mg, about 10 mg to about 70 mg, about 10 mg to about 60 mg, about 10 mg to about 50 mg, about 10 mg to about 40 mg, about 10 mg to about 30 mg, about 10 mg to about 20 mg, from about 20 mg to about 450 mg, from about 20 mg to about 400 mg, from about 20 mg to about 350 mg, from about 20 mg to about 300 mg, from about 20 mg to about 250 mg, from about 20 mg to about 200 mg, from about 20 mg to about 175 mg, from about 20 mg to about 150 mg, from about 20 mg to about 125 mg, from about 20 mg to about 100 mg, from about 20 mg to about 90 mg, from about 20 mg to about 80 mg, from about 20 mg to about 70 mg, from about 20 mg to about 60 mg, from about 20 mg to about 50 mg, from about 20 mg to about 40 mg, from about 20 mg to about 30 mg, from about 50 mg to about 450 mg, from about 50 mg to about 400 mg, from about 50 mg to about 350 mg, from about 50 mg to about 300 mg, from about 50 mg to about 250 mg, from about 50 mg to about 200 mg, from about 50 mg to about 175 mg, from about 50 mg to about 150 mg, from about 50 mg to about 125 mg, from about 50 mg to about 100 mg, from about 50 mg to about 90 mg, from about 50 mg to about 80 mg, from about 50 mg to about 70 mg, from about 50 mg to about 60 mg, from about 100 mg to about 450 mg, from about 100 mg to about 400 mg, from about 100 mg to about 350 mg, from about 100 mg to about 300 mg, from about 100 mg to about 250 mg, from about 100 mg to about 200 mg, from about 100 mg to about 175 mg, from about 100 mg to about 150 mg, from about 100 mg to about 125 mg, or about 1, 2, 3, 4, 5, 6, 7, 8, 9, 10, 15, 20, 25, 30, 35, 40, 45, 50, 55, 60, 65, 70, 75, 80, 85, 90, 95, or 100 mg.

In the pharmaceutical combination, examples of the amount or dose of the arginine or N-acetylcysteine used herein include, but are not limited to, from about 10 mg to about 2 g, from about 10 mg to about 1.9 g, from about 10 mg to about 1.8 g, from about 10 mg to about 1.7 g, from about 10 mg to about 1.6 g, from about 10 mg to about 1.5 g, from about 10 mg to about 1.4 g, from about 10 mg to about 1.3 g, from about 10 mg to about 1.2 g, from about 10 mg to about 1.1 g, from about 10 mg to about 1.0 g, from about 10 mg to about 900 mg, from about 10 mg to about 800 mg, from about 10 mg to about 700 mg, from about 10 mg to about 600 mg, from about 10 mg to about 500 mg, from about 10 mg to about 400 mg, from about 10 mg to about 300 mg, from about 10 mg to about 200 mg, from about 10 mg to about 100 mg, from about 10 mg to about 50 mg, from about 10 mg to about 25 mg, from about 100 mg to about 2 g, from about 100 mg to about 1.9 g, from about 100 mg to about 1.8 g, from about 100 mg to about 1.7 g, from about 100 mg to about 1.6 g, from about 100 mg to about 1.5 g, from about 100 mg to about 1.4 g, from about 100 mg to about 1.3 g, from about 100 mg to about 1.2 g, from about 100 mg to about 1.1 g, from about 100 mg to about 1.0 g, from about 100 mg to about 900 mg, from about 100 mg to about 800 mg, from about 100 mg to about 700 mg, from about 100 mg to about 600 mg, from about 100 mg to about 500 mg, from about 100 mg to about 400 mg, from about 100 mg to about 300 mg, from about 100 mg to about 200 mg, or about 2, 1.9, 1.8, 1.7, 1.6, 1.5, 1.4, 1.3, 1.2, 1.1, or 1.0 g, or about 950, 900, 850, 800, 750, 700, 650, 600, 550, 500, 450, 400, 350, 300, 250, 200, 150, 100, 50, 25, or 10 mg.

The combination or composition disclosed herein generally includes one or more pharmaceutically acceptable excipients, carriers or diluents. The particular carrier, diluent or excipient used will depend upon the means and purpose for which the active ingredient is being applied. In general, a tablet formulation includes materials such as diluents, binders, lubricants, disintegrants and mixtures thereof. Suitable diluents include various types of starch, lactose, mannitol, kaolin, calcium phosphate or sulfate, inorganic salts (e.g., sodium chloride), powdered sugar, and powdered cellulose derivatives. More specifically, examples of diluents or fillers include lactose, mannitol, xylitol, dextrose, sucrose, sorbitol, compressible sugar, microcrystalline cellulose, powdered cellulose, starch, pregelatinized starch, dextrates, dextran, dextrin, dextrose, maltodextrin, calcium carbonate, dibasic calcium phosphate, tribasic calcium phosphate, calcium sulfate, magnesium carbonate, magnesium oxide, poloxamers such as polyethylene oxide and hydroxypropyl methyl cellulose. If desired, a binder may be added. Suitable binders include substances such as celluloses (e.g., cellulose, methylcellulose, ethylcellulose, and hydroxymethylcellulose), polypropylpyrrolidone, polyvinylpyrrolidone, gelatin, gum arabic, polyethylene glycol, starch, sugars (e.g., lactose, sucrose, fructose, and glucose), natural and synthetic gums (e.g., acacia, alginates, and gum arabic) and waxes. A lubricant is typically used in a tablet formulation to prevent the tablet and punches from sticking in the die. Suitable lubricants include calcium stearate, glyceryl monostearate, glyceryl palmitostearate, hydrogenated vegetable oil, light mineral oil, magnesium stearate, mineral oil, polyethylene glycol, sodium benzoate, sodium lauryl sulfate, sodium stearyl fumarate, stearic acid, talc and zinc stearate. Disintegrants may also be added to the combination or composition to break up the dosage form and release the compound. Suitable disintegrants include sodium starch glycolate, sodium carboxymethyl cellulose, calcium carboxymethyl cellulose, croscarmellose sodium, polyvinylpyrrolidone, methyl cellulose, microcrystalline cellulose, powdered cellulose, lower alkyl-substituted hydroxypropyl cellulose, polacrilin potassium, starch, pregelatinized starch and sodium alginate.

The combination or composition of a PDE5 inhibitor in combination, arginine and NAC is useful in the treatment of a vascular disease. Surprisingly, a combination or composition of NAC and arginine can enhance the effect of a PDE5 inhibitor and simultaneously reduce adverse effects of the PDE5 inhibitor. Accordingly, the present disclosure provides a method for treating a vascular disease, comprising administering an effective amount of the pharmaceutical combination or composition of a PDE5 inhibitor, arginine and NAC to a subject. The present disclosure also provides a method for treating a disease associated with inhibition of PDE5 while reducing adverse effects of the PDE5 inhibition, comprising administering an effective amount of NAC and arginine in combination with a PDE5 inhibitor.

Examples of the vascular disease include, but are not limited to, erectile dysfunction, mountain sickness, pulmonary hypertension, vascular fibrosis, and vascular sclerosis.

The beneficial effect of the combination includes, but is not limited to, pharmacokinetic and/or pharmacodynamic co-action resulting from the combination of said agents. Administration of these agents in combination typically is carried out over a defined time of period (usually minutes, hours, days or weeks depending upon the combination selected). Combination therapy is intended to embrace administration of the indicated therapeutic agents in a sequential manner, that is, wherein each therapeutic agent is administered at a different time, as well as administration of these therapeutic agents, in a substantially simultaneous manner. Administration can be accomplished, for example, by administering to the subject a single oral dosage form having a fixed ratio of each agent or in multiple, single oral dosage forms for each of the therapeutic agents. Sequential or substantially simultaneous administration of each therapeutic agent can be effected by any appropriate route including, but not limited to, oral routes, intravenous routes, intramuscular routes, and direct absorption through mucous membrane tissues.

In another embodiment, the combinations and compositions of the present disclosure can be administered parenterally (e.g., by intramuscular, intrathecal, intravenous, and intraarterial routes), preferably, intravenously. Typically, compounds and compositions of the invention for intravenous administration are solutions in sterile isotonic aqueous vehicles, such as water, saline, Ringer's solution, or dextrose solution. Where necessary, the compositions may also include a solubilizing agent. The combinations and compositions for intravenous administration may optionally include a local anesthetic such as lignocaine to ease pain at the site of the injection. For intravenous administration, the combinations and compositions of the present disclosure can be supplied as a sterile, dry lyophilized powder or water-free concentrate in a hermetically sealed container, such as an ampule or sachette, the container indicating the quantity of the active agent. Such a powder or concentrate is then diluted with an appropriate aqueous medium prior to intravenous administration. An ampule of sterile water, saline solution, or other appropriate aqueous medium can be provided with the powder or concentrate for dilution prior to administration. Or the combinations and compositions of the present disclosure can be supplied in pre-mixed form, ready for administration. Where the combinations and compositions of the present disclosure are to be administered by intravenous infusion, they can be dispensed, for example, with an infusion bottle containing sterile pharmaceutical-grade water, saline, or another suitable medium. Rectal administration can be effected through the use of suppositories formulated from conventional carriers such as cocoa butter, modified vegetable oils, and other fatty bases. Suppositories can be formulated by well-known methods using well-known formulations; for example, see Remington: The Science and Practice of Pharmacy, Alfonso R. Gennaro ed., Mack Publishing Co. Easton, Pa., 19th ed., 1995, pp. 1591-1597, incorporated herein by reference

A therapeutically effective dosage regimen for the treatment of a particular disorder or condition will depend on its nature and severity, and can be determined by standard clinical techniques according to the judgment of a medical practitioner. In addition, in vitro or in vivo assays can be used to help identify optimal dosages. Of course, the amount of a compound of the invention that constitutes a therapeutically effective dose also depends on the administration route.

The following examples are offered by way of illustration and are not intended to limit the scope of the invention.

### EXAMPLES

### Experimental Methods

### Preparation of Drug

Sildenafil, arginine, N-acetylcysteine, and S-nitroso-N-acetylpenicillamine (SNAP) are dissolved and diluted in phosphate buffered saline (PBS). In particular, the following solutions are prepared: a solution containing 100 nM sildenafil (hereinafter called SILD or “S”); a mixed solution of 100 nM of sildenafil and 10 mM of N-acetylcysteine (hereinafter called “SN”); a mixed solution of 100 nM of sildenafil, 150 μM of arginine and 10 mM of N-acetylcysteine (hereinafter called “SAN”); a mixed solution of 100 nM of sildenafil and 100 μM S-nitroso-N-acetylpenicillamine (SNAP) (hereinafter called “SS”); and a mixed solution of 100 nM of sildenafil, 100 μM SNAP, and 10 mM of N-acetylcysteine (hereinafter called “SSN”). The solutions are stored at a temperature of 4° C. For clinical trials and human test, the combination of SAN comprising 20 mg of sildenafil, 250 mg of arginine, and 250 mg of NAC is used and applied to subjects.

### Cell Culture

Human umbilical vein endothelial cells (HUVEC) obtained from Bioresource Collection and Research Center (Hsinchu, Taiwan) were cultured in Ham's F-12K (Sigma-Aldrich) with endothelial cell growth supplement (Millipore), heparin (Sigma-Aldrich), 2.2 mg/mL sodium bicarbonate, and 10% FBS (Gibco). HUVEC at passage 5 to 15 were used in all applications. Human pulmonary artery vascular smooth muscle (PAVSM) cells and growth media were obtained from Lonza Inc. (Walkersville, MD., USA). Cells were grown in a 5% CO2 atmosphere at 37° C.

### Cell Treatment

For cell treatments, HUVEC and PAVSM cells were grown in confluence respectively on the upper and lower wells of Transwell® units. Culture medium volume in each upper chamber was 1.5 mL, and in each lower chamber was 2.5 mL. Confluent HUVEC cells were treated with regular medium in the presence or absence of S, SS, SN, SSN and SAN for 0, 10, 20, 30 and 60 min. All supernatants of HUVEC and PAVSM were collected right after the treatments for the NO amount analysis. HUVEC cells and PAVSM cells were rinsed in ice-cold phosphate buffered saline and then solubilized in ice-cold 6% trichloroacetic acid for the protein extraction. Samples were stored at −80° until the assays.

Analysis of Nitric Oxide Release Amount from Endothelial Cells

The nitric oxide amount was assessed using the NO assay kit (Promega). After treatment, the supernatants of HUVEC were collected for the NO assay reagent kit, and 200 μL per well was applied according to the manufacturer's instructions. Relative Luminance Unit (RLU) emitted by the product was measured using a microreader.

### Analysis of Expression Amount of Endothelial Nitric Oxide Synthase

Endothelial nitric oxide synthase (eNOS) activity was assessed using the eNOS assay kit (Promega). After treatment, the supernatant plates were removed from the incubator and allowed to equilibrate to room temperature for approximately 30 min, and then the eNOS assay reagent (200 μL/well) was applied according to the manufacturer's instructions. Relative Luminance Unit (RLU) emitted by the product was measured using a microreader.

Analysis of cGMP in Smooth Muscle Cells

Samples subjected to drug treatment for 0, 10, 20, 30 and 60 min were processed and cGMP and protein were measured as described previously (Garmaroudi et al., Systems Pharmacology and Rational Polypharmacy: Nitric Oxide-Cyclic GMP Signaling Pathway as an Illustrative Example and Derivation of the General Case; PLOS COMPUTATIONAL BIOLOGY, DOI:10.1371/journal.pcbi.1004822 Mar. 17, 2016). cGMP formation was measured by immunoassay according to the cGMP Assay (Cayman Chemical Co., Ann Arbor, MI).

### Detection Mode of Quantitative Free Radical ROS Content

ROS detection studies were performed using a Cm-H₂DCFDA ROS detection kit (Invitrogen, Grand Island, NY, USA). For cell-free ROS detection, aliquots of 100 μL serum-free medium containing each concentration of PM2.5 was pipetted into black 96-well plates and mixed with 10 μL Hanks' Balanced Salt Solution (HBSS) containing Cm-H₂DCFDA (final concentration 25 μM), activated by pre-incubation at 37° C. for 30 min. ROS generation was measured every 10 min up to 2 h with a micro-reader at 490 nm excitation, 530 nm emission. The values were presented as RFU. For detection of intracellular ROS, HUVECs were incubated with PM2.5 for 24 h, after which they were washed with PBS and treated with fresh serum-free medium containing Cm-H₂DCFDA (final concentration 25 μM). Samples were then measured immediately using a Promega microreader. ROS levels generated by 2.5×10⁴ viable treated cells were expressed as the percentage of ROS produced by an equal number of viable negative control cells.

### Vascular Fibrosis Index Analysis: Detection of Free Radical-Induced Lipid Peroxidation (Malondialdehyde, MDA)

Lipid peroxidation in HUVEC protein extracts was quantified measuring the concentration of MDA by a spectrofluorometric assay using an “MDA assay kit” as described by Richard et al., 1992 (Richard et al. 1992). Quantification was achieved by parallel measurements of a standard curve of known MDA concentrations, and results were expressed as level change of the control.

### Vascular Permeability Analysis: VEGF-A Release Content

VEGFA amount was assessed using the VEGFA assay kit (Promega). After treatment, the plates were moved from the incubator and allowed to equilibrate to room temperature for approximately 30 min, and then the VEGFA assay reagent (200 μL/well) was applied according to the manufacturer's instructions. Relative Absorbance Unit at 540 nm emitted by the product was measured using a microreader.

### Test for the Efficacy of SAN in Human

A human/clinical study is conducted in which patients with erectile dysfunction are treated with a combination of SAN comprising 20 mg of sildenafil, 250 mg of arginine and 250 mg of NAC. Pharmacokinetics and Pharmacodynamics of SAN as well as the efficacy of SAN, including the efficacy in treating ED and vascular diseases, are assayed. The evaluation indexes for ED and vascular diseases known in the art are applied herein, including the assays for NO, NOS, ROS, cGMP, and indexes for vascular diseases described herein or known in the art. The subjects administered the SAN combination are between the ages of 19 and 87. Regarding ED, the curative effect of SAN is assessed by the worldwide assessment questionnaire, daily erection record, International Index of Erectile Function (IIEF, a validated sexual function questionnaire) and/or questionnaire survey of sexual partners.

### Experimental Results

### Example 1. The Combination SAN Increases the Reaction Rate of Sildenafil and Decreases the Reaction Time of Sildenafil

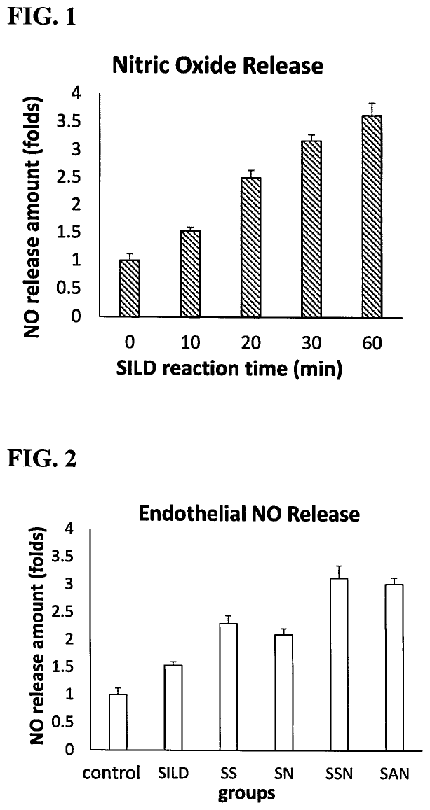

FIG. 1 is a scale chart showing the relationship between the NO release amount and treatment time of vascular endothelial cells treated by the drug or drug combination as disclosed herein. As shown in FIG. 1, the release amount of nitric oxide is increased along with the treatment time of SILD. Compared with the control group (drug treatment for 0 min), the release amount of nitric oxide reaches about 1.6 folds at the time point after drug treatment for 10 min, reaches about 2.6 folds at the time point after drug treatment for 20 min, reaches about 3.1 folds at the time point after treatment for 30 min and reaches about 3.5 folds at the time point after treatment for 60 min. FIG. 2 is a scale chart of the nitric oxide release amount of vascular endothelial cells after the vascular endothelial cells are treated with SILD, SN and SAN for 10 min. As shown in FIG. 2, after a 10-min drug treatment, compared with the control group (with no drug; taken as a reference), the release amount of nitric oxide of the SILD group(S) reaches about 1.6 folds, that of the SN group reaches about 2 folds, and that of the SAN group reaches about 3.1 folds. The results show that only a 10-min treatment with SAN can achieve the same NO release effect as a 30-min treatment with SILD; see FIG. 1 and FIG. 2. That is, the reaction rate of SILD in the combination SAN is faster than SILD alone.

### Example 2. The Combination SAN Increases the Expression of Nitric Oxide Synthase

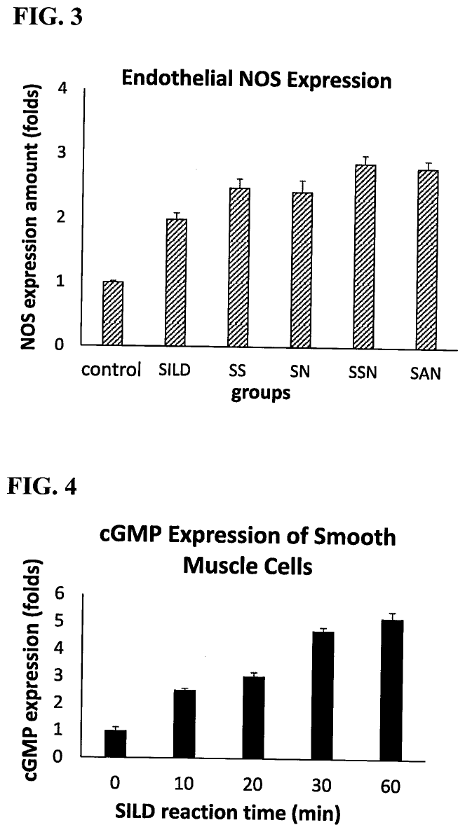

FIG. 3 is a scale chart of the expression amount of vascular endothelial nitric oxide synthase after the vascular endothelial cells are treated with SILD, SN and SAN for 10 min; SS and SSN were also used for treatment (results not shown in FIG. 3). Compared with the control group (with no drug; taken as a reference), the expression amount of the nitric oxide synthase of the SILD(S), SS, SN, and SAN groups reaches about 2 folds, 2.5 folds, 2.4 folds, and 2.8 folds, respectively. According to the results, the expression amount of endothelial nitric oxide synthase in treatment with SAN is obviously higher than that in treatment with only SILD; see FIG. 3.

### Example 3. The Combination SAN Increases the Effect and Reaction Rate of Sildenafil, and Decreases the Reaction Time of Sildenafil

FIG. 4 is a scale chart of a relationship between the cGMP expression amount of smooth muscle cells and treatment time in treatment of the smooth muscle cells with Sildenafil (SILD). The cGMP content of the smooth muscle cells reflects the degree of vasodilatation; as shown in FIG. 4, the amount of the cGMP increases along with the action time of treatment with SILD. Compared with the control group (drug treatment for 0 min; taken as a reference), the amount of cGMP reaches about 2.6 folds, 3 folds, 4.7 folds, and 5.5 folds after drug treatment for 10, 20, 30, and 60 min, respectively.

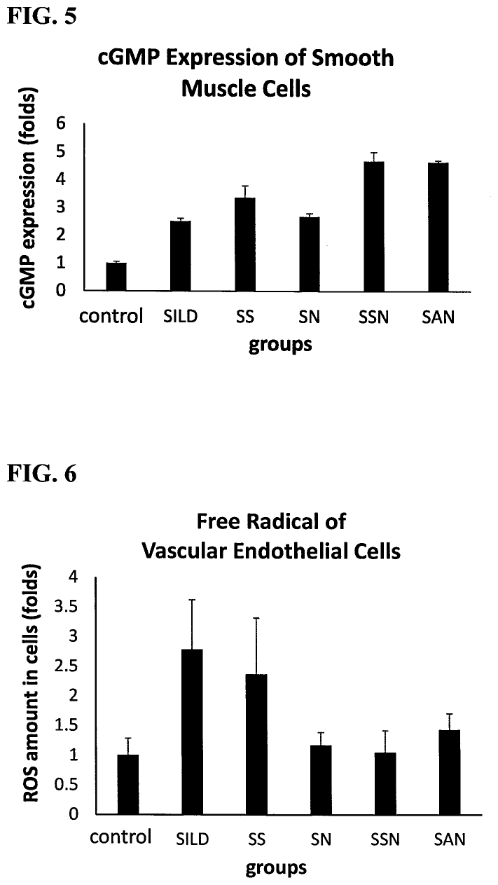

FIG. 5 is a scale chart of the cGMP expression amount of smooth muscle cells after the smooth muscle cells are treated with SILD, SN and SAN for 10 min. As shown in FIG. 5, after a 10-min treatment, compared with the control group (with no drug; taken as a reference), the amount of cGMP reaches about 2.6 folds in the SILD(S) group, reaches about 2.7 folds in the SN group, and reaches about 4.6 folds in the SAN group. The results show that after the cells are treated with SILD, the amount of cGMP of the smooth muscle cells is increased; when the cells are treated with SAN, only 10 min of treatment can achieve the same effect as 30 min of treatment with SILD (see FIG. 4 and FIG. 5). That is, the reaction rate of the drug in treatment with SAN is faster than that in treatment with only SILD.

### Example 4. The Combination SAN Decreases the Vascular Layer Fibrosis Indexes and Vascular Permeability Caused by Sildenafil

FIG. 6 is a scale chart showing vascular endothelial cell free radical content after vascular endothelial cells are treated with SILD, SN and SAN for 10 min. After treatment with SILD, vascular endothelial cells bear the pressure from high blood flow due to rapid vasodilatation caused by SILD, which promotes the vascular endothelial cells to release endogenous free radicals. FIG. 6 shows that the amount of ROS in the cells changes along with the treatment with different drugs. Compared with the control group (with no drug treatment; taken as a reference), the amount of ROS reaches about 2.8 folds in the SILD(S) group, and reaches about 1 fold in the SN and SAN groups. The results show that the amount of the ROS produced in treatment with SILD can be effectively reduced by the combination disclosed herein, in particular NAC in the combination.

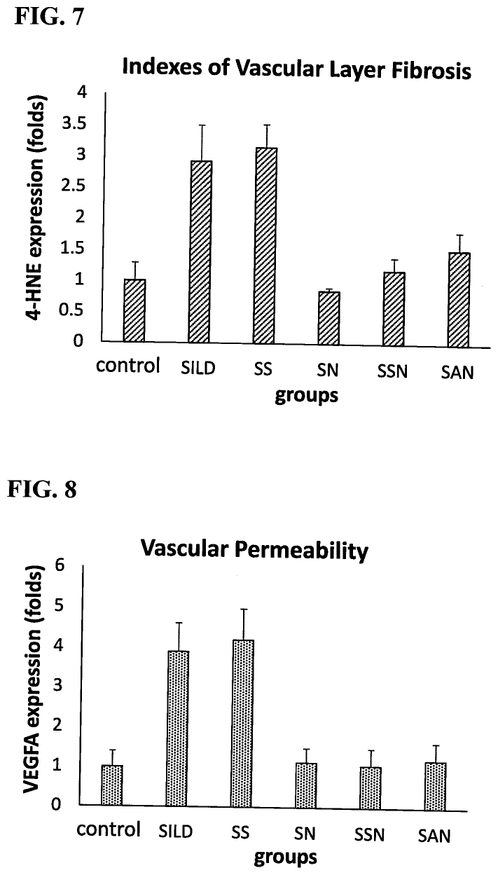

FIG. 7 is a scale chart showing vascular layer fibrosis indexes after vascular endothelial cells are treated with SILD, SN and SAN for 10 min. Increased endogenous free radicals cause peroxidation of cell lipids and further cause vascular fibrosis lesions. FIG. 7 shows that the amount of malondialdehyde (MDA) in the cells changes after the treatment with different drugs. Compared with the control group (with no drug treatment; taken as a reference), the amount of MDA reaches about 2.8 folds the SILD group, and reaches about 1 fold in the SN and SAN groups. The results show that the amount of the MDA results from SILD is effectively reduced by the combination disclosed herein, in particular NAC in the combination.

FIG. 8 is a scale chart showing vascular permeability after vascular endothelial cells are treated with SILD, SN, and SAN for 10 min. The local increase of vascular permeability is one of indexes of vascular fibrosis and vascular sclerosis, and the increase of vascular permeability factor VEGFA (vascular endothelial growth factor A) causes vascular fibrosis and vascular sclerosis. FIG. 8 shows that the amount of VEGFA in the cells changes along with the treatment with different drugs. Compared with the control group (with no drug treatment; taken as a reference), the amount of VEGFA reaches about 3.9 folds in the SILD group(S), and reaches about 1 fold in the SN and SAN groups. The results show that the amount of the VEGFA caused by the treatment with SILD is effectively reduced by the combination disclosed herein, in particular NAC in the combination.

### Example 5

A human/clinical study is conducted in which patients with erectile dysfunction are treated with a combination of SAN comprising 20 mg of sildenafil, 250 mg of arginine and 250 mg of NAC. The time at which the highest blood concentration can be observed when subjects take a combination of SAN orally in a fasting state is determined; past data show that sildenafil alone is rapidly absorbed after administration, (the highest blood concentration can be observed within 30 to 120 minutes (median 60 minutes)), and the average absolute bioavailability of oral administration is ranged between 25 to 63%. The clearance rate of sildenafil is observed especially for healthy elderly subjects (65 years or older); past data showed that after administration of sildenafil alone, the clearance rate in elderly subjects decreased, and the free plasma concentration thereof is 40% higher than in younger subjects (18-45 years). The efficacy and safety of SAN is further evaluated in randomized, double-blind, placebo-controlled trials. The subjects administered the SAN combination are between the ages of 19 and 87, and the patients have erectile dysfunction due to different etiologies (organic, psychogenic or mixed). The curative effect of SAN is then assessed by the worldwide assessment questionnaire, daily erection record, International Index of Erectile Function (IIEF, a validated sexual function questionnaire) and questionnaire survey of sexual partners. Past data show that the proportions of disease improvement after taking sildenafil alone are 62% (25 mg), 74% (50 mg) and 82% (100 mg), respectively, as for the placebo, only 25%. According to relevant preliminary test results, the inventor believes that the SAN combination can achieve the same efficacy as that of known drugs with higher doses, faster response/reaction rate and/or fewer side effects.

According to the results, the SAN combination disclosed herein can obviously reduce the amount of ROS produced after the smooth muscle cells are treated with SILD, reduce the cell lipid peroxidation caused by ROS and the VEGFA, and further reduce abnormally increased vascular permeability and the risk of vascular sclerosis.

In addition, the SAN combination disclosed herein surprisingly increases the reaction rate of SILD, reduces the reaction time, and scavenges redundant free radicals in the cells so as to achieve the functions of protecting blood vessels and preventing blood vessel walls from fibrosis and sclerosis after long-term use. Therefore, the combination or composition disclosed herein for treating vascular diseases can be taken long term with fewer side effects.

The above description and illustration only represent a description of preferred embodiments of the present invention. Those skilled in the art can make modifications according to the general knowledge and the present invention herein, but these modifications should still be within the scope of the present invention herein for the spirit of the present invention.

What is claimed is:

1. A pharmaceutical combination comprising a phosphodiesterase type 5 (PDE5) inhibitor or a pharmaceutically acceptable salt thereof, arginine, and N-acetylcysteine; optionally, the pharmaceutical combination is in the form of a pharmaceutical composition comprising a pharmaceutically acceptable excipient.

2. The pharmaceutical combination of claim 1, wherein the PDE5 inhibitor is selected from sildenafil, tadalafil, and vardenafil.

3. The pharmaceutical combination of claim 1, wherein
the amounts of the PDE5 inhibitor, arginine and N-acetylcysteine range from about 0.1% (w/w) to about 50% (w/w), about 4.0% (w/w) to about 80% (w/w) and about 4% (w/w) to about 80% (w/w), respectively; or
the pharmaceutical combination comprises about 0.5 mg to about 250 mg of the PDE5 inhibitor, about 25 mg to about 400 mg of arginine and about 25 mg to about 400 mg of N-acetylcysteine.

4. The pharmaceutical combination of claim 1, which further comprises one or more preservatives selected from the group consisting of an antibacterial agent, an antifungal agent, and an antiseptic.

5. The pharmaceutical combination of claim 1, which is in the form of a solid or liquid formulation.

6. The pharmaceutical combination of claim 5, which is a capsule or a tablet.

7. A method for treating a vascular disease comprising administering the pharmaceutical combination of claim 1 to a subject in need thereof.

8. A method for treating a disease associated with inhibition of PDE5 while reducing adverse effects of the PDE5 inhibition comprising administering a pharmaceutical combination comprising an effective amount of N-acetylcysteine and arginine in combination with a PDE5 inhibitor or a pharmaceutically acceptable salt thereof to a subject in need thereof.

9. The method of claim 8, wherein the disease associated with inhibition of PDE5 is a vascular disease selected from erectile dysfunction, mountain sickness, pulmonary hypertension, vascular fibrosis, and vascular sclerosis.

10. The method of claim 8, wherein the PDE5 inhibitor is sildenafil, and wherein the combination increases the reaction rate of sildenafil or decrease the reaction time of sildenafil, compared with administration of sildenafil alone.

11. The method of claim 8, wherein the combination significantly increases the expression of nitric oxide synthase in a cell.

12. The method of claim 8, wherein the PDE5 inhibitor or a pharmaceutically acceptable salt, arginine, and N-acetylcysteine is administered concomitantly, sequentially, or separately.

13. The method of claim 8, wherein the PDE5 inhibitor is sildenafil or sildenafil citrate.

14. The method of claim 8, wherein the combination is administered twice a day.
